(19) United States
(12) Patent Application Publication
(10) Pub. No.: US 2005/0119559 A1
(43) Pub. Date: Jun. 2, 2005

(54) Diagnostic apparatus with an automatic visualization of scan planes

(75) Inventors: Johannes Jacobus Van Vaals, Eindhoven (NL); Tatiana Borisovna Sazonova, St. Petersburg (RU)

Correspondence Address: Thomas M Lundin;Philips Intellectual Property & Standards, 595 Miner Road, Cleveland, OH, 44143, US

(73) Assignee: Koninklijke Philips Electronics N.V, BA Eindhoven (NL)

(21) Appl. No.: 10/505,233

(22) PCT Filed: Jan. 20, 2003
(86) PCT No.: PCT/IB03/00128

(30) Foreign Application Priority Data
Feb. 20, 2002 (EP) 02075682.1

(51) Int. Cl. A61B 5/05

(52) U.S. Cl. 600/425

(57) ABSTRACT

A diagnostic apparatus (1) according to the invention is arranged to comprise imaging means (6) for acquiring diagnostic information within a volume of a patient (P) being located with an imaging volume (1′) of the diagnostic apparatus (1). In order to visualise the spatial position and orientation of an actual scanning plane on the skin of the patient corresponding to an actual diagnostic image, the diagnostic apparatus (1) comprises visualisation means (10,11,12). The visualisation means (10,11,12) are arranged in an immediate vicinity of the imaging volume (1′) and can be realised as a set of light fan beams. The correct position of the light fans with respect to the actual imaging plane can be mechanically adjusted or can be adjusted by means of a mirror-based optical arrangement.

The invention relates to a diagnostic apparatus comprising an imaging volume for accommodating a patient to be imaged, means for positioning the patient within the imaging volume, imaging means arranged to acquire a diagnostic image in an imaging plane of the patient positioned in the imaging volume.

The invention further relates to a method for guiding an interventional apparatus using the diagnostic apparatus.

An apparatus to perform diagnostic studies by means of a spin magnetic resonance of a patient located in the imaging volume of said apparatus is known from U.S. Pat. No. 6,275,721. The known apparatus is further equipped with a visual feedback to provide an information about a sighting axis towards the imaging plane. This information is provided by a visualization of an impingement point of said axis on the patient's skin by means of a visible laser diode.

The known apparatus has a disadvantage that no information about the orientation of the scan plane is provided for analysis by the operator. In order to find the entry lines of the scan plane on the surface of the patient the operator has to reconstruct the spatial orientation of the actual plane. This is a very difficult and unreliable procedure as it is based upon the diagnostic image comprising a target area. For oblique scan planes this may lead to a very inaccurate result. Thus, provided with only a given projection of the sighting axis on the surface of the patient it is not feasible for the operator to deduct the entry lines of the actual scan plane, which is particularly important for conducting interventional procedures.

It is an object of the invention to provide an improved diagnostic apparatus.

The diagnostic apparatus according to the invention is characterized in that the diagnostic apparatus further comprises visualization means arranged to visualize a spatial position of the imaging planes within the imaging volume. According to the technical measure of the invention the operator is provided with a tangible information about the projection of the scanning plane onto the patient. This information can be used during a therapy planning procedure, where the exact location of the entry lines of the scan plans must be known in order to plan a subsequent radiotherapy. The technical measure of the invention is also advantageous for planning follow-up examinations, where the patient must be scanned in exactly the same scanning planes as during a previous examination. By positioning the patient in such a way in the imaging volume that the actual scan planes spatially coincide with the marked previous plane a greater reliability of the follow-up consistency can be guaranteed. Furtheron, the technical measure of the invention can also be applied in the field of medical interventions, where an examination apparatus, or a biopsy needle has to be inserted into the patient with a high spatial accuracy. By visualizing the entry lines of the scan planes on the skin of the patient the position of the incision can be controlled better. Furtheron, this technical measure simplifies the procedure for bringing markers for representing an internal lesion on the patient's skin. It must be noted that the technical measure of the invention can be integrated in a wide variety of medical diagnostic apparatus, for example an X-ray apparatus, an MRI apparatus or a computer tomograph.

An embodiment of the invention is characterized in that the visualization means are arranged in the immediate vicinity of the imaging volume and in that the visualization means comprise an adjustable light fan. By providing a plurality of light sources with fan-like bundles a spatial position of a plane can be visualized. Preferably, the light sources are located within a bore of a bore-type apparatus, or are mounted on a foot- or ceiling based arm for a conventional X-ray apparatus. The correct position of the light fans can be adjusted mechanically or using mirrors, which are controlled by the unit controlling the orientation of the scan planes. In this way a direct link between the diagnostic information and an external anatomy of the patient is obtained.

A further embodiment of the apparatus according to the invention is characterized in that the visualization means further comprise indicators to visualize a selected area within the imaging plane. An example of such a selected area can be a center of the plane. Alternatively, the operator can indicate with a cursor an incision position on the diagnostic image and the visualizing means can be arranged to visualize the selected incision point of the surface of the patient. Due to this technical measure the guiding of a medical instrument during the interventional procedures can be performed with an increased reliability.

A method according to the invention is characterized in that said method comprising the steps of positioning a patient within the imaging volume of the diagnostic apparatus; using the imaging means for acquiring a diagnostic image in a plane comprising a target area of the patient; using the visualization means for visualizing a projection of the imaging plane of said diagnostic image on the patient's skin. By applying the method according to the invention the operator is provided with an accurate information about the spatial relation of the plane of the diagnostic image comprising a target area, for instance a lesion and the external anatomy of the patient. In this way a direct link is provided between the location of internal anatomy, target areas and the external world. This information is of particular importance for interventional applications.

A further method according to the invention is characterized in that said method comprises the steps of: positioning a patient within the imaging volume of the diagnostic apparatus; using the imaging means for acquiring a diagnostic image in a plane comprising a target area of the patient; calculating an approach trajectory for the interventional apparatus, said trajectory comprising an entry point on the patient's skin and a target point within the target area; visualizing the entry point together with a projection of the imaging plane of the diagnostic image on the patient's skin. The method according to the invention is particular valuable for guiding the interventional procedures. It is well known, that for some applications, for example for cranial interventions, it is of vital importance not to damage certain areas of a healthy tissue. By applying the method according to the invention the operator can select a target area on the acquired diagnostic image, and the system can provide the optimal approach trajectory including an entry point on the skin surface of the patient and the selected point within the target area. When the optimal trajectory is calculated, the location of the entry point can be visualized on the skin of the patient together with the projection of the plane of the diagnostic image. It is also possible to add an additional visual guide to simplify the determination of the angular orientation of the interventional apparatus with respect to the entry point.

These and other aspect of the invention will be discussed in more detail with reference to figures.

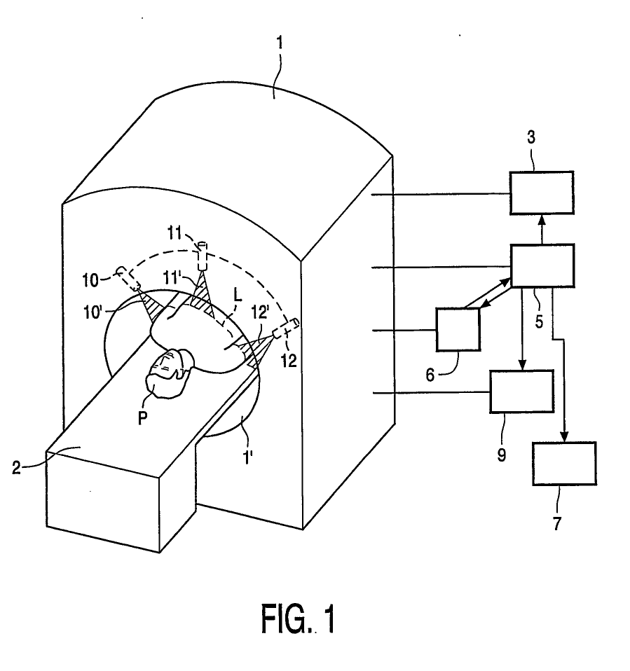

FIG. 1 presents a schematic view of an embodiment of the diagnostic apparatus according to the invention.

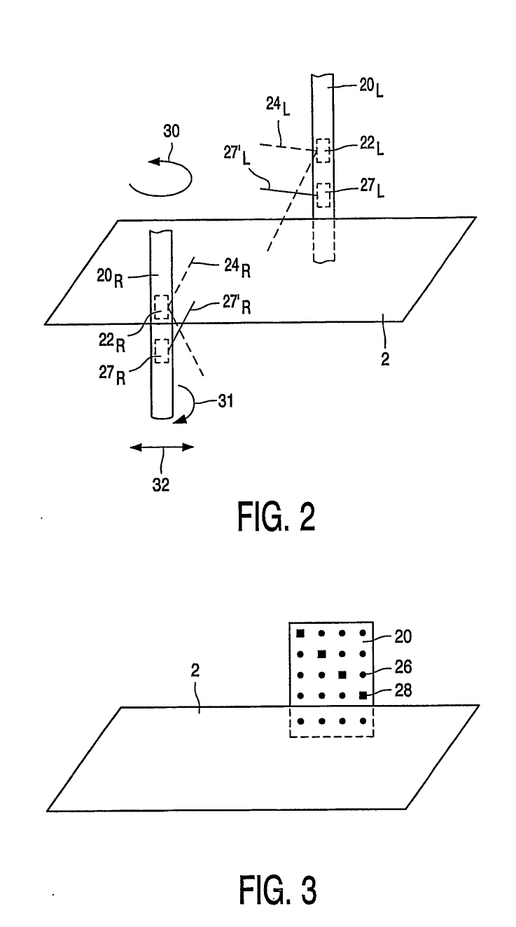

FIG. 2 shows schematically a first embodiment of the visualization means according to the invention.

FIG. 3 shows schematically a second embodiment of the visualization means according to the invention.

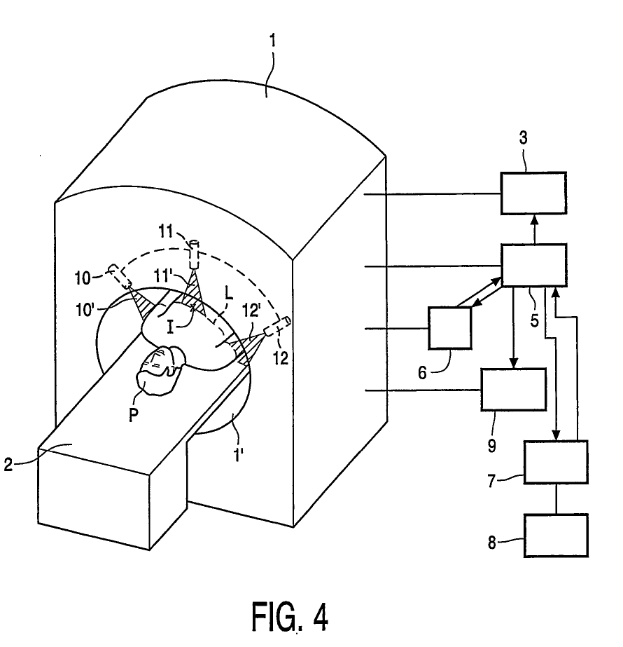

FIG. 4 shows schematically an embodiment of the diagnostic apparatus arranged to carry out the method according to the invention.

FIG. 1 shows schematically an embodiment of the diagnostic apparatus according to the invention. In this figure the diagnostic apparatus 1 is a conventional bore-type magnetic resonance apparatus comprising a support table 2 which is movable to arrange a patient P to be examined an imaging volume 1′ of the diagnostic apparatus 1. The operational principle of such an apparatus is known, for example from WO 98/10303 and lies within the scope of knowledge of the person skilled in the art. A central control unit 5 of the MR-apparatus 1 is arranged to control gradient coils (not shown) by sending an appropriate signal to a respective coil control system 3 defining each component Gx, Gy and Gz of the gradient field. The imaging means 6 are arranged to acquire a diagnostic image from the imaging plane defined by the gradient field. The spatial position and orientation of the imaging plane is unambiguously defined when all three components Ox, Gy and Gz of the gradient field are defined. Therefore, by using this information it is possible to unambiguously define a visual plane representing the imaging plane of the diagnostic image in the imaging volume. This is carried out in the diagnostic apparatus according to the invention by means of a control unit 9 which is controlled by the central control unit 5 in coherence with the control unit 3 of the gradient coils. The actual visualization of the imaging plane is performed by means of light fans 10′, 11′, 12′ propagating in the imaging volume 1′ from the light-emitting diodes 10, 11, 12 arranged in the bore of the diagnostic apparatus 1. A line L representing an intersection of a light fan with the surface of the patient represents the projection of the imaging plane on the patient's skin. The operator is thus provided with the visualization of the spatial position of the imaging plane together with the diagnostic data presented on the console 7. The visualization means can be realized for example by light sources emitting light fans, the correct position of the fans with respect to the imaging plane being mechanically adjusted or being adjusted by means of a mirror-based optical arrangement.

FIG. 2 shows schematically a first embodiment of the visualization means according to the invention. The visualization means 20l, 20r can be arranged in an immediate vicinity of the patient table 2, preferably in the imaging volume (not shown in FIG. 2). Alternatively, the visualization means 20r, 201 can be arranged in the bore of the diagnostic apparatus. This can easily be implemented especially for so-called open MR-scanners. The embodiment shown in FIG. 2 comprises an assembly of a two floor-mounted arms, each arm bearing a light fan source 22l, 22r, respectively, emitting visible light in a fan geometry, indicated by 24l, 24r. In general, the systems conventionally suited for the patient positioning and alignment are well suited to be used as visualization means within the teaching of the present invention. In order to visualize an oblique plane, each arm is rotatable around three orthogonal axis, as schematically is indicated by arrows 30, 31, 32 in FIG. 2. The rotation of the arm is controlled by a conventional drive (not shown) in accordance with a signal from the control unit 9 (see FIG. 1). The visualization means 20l and 20r can be further equipped with indicators 27l, 27r in order to visualize a selected area on the surface of the patient P. This can be realized in case the indicators 27l and 27r emit visible light in a substantially pencil-bean geometry, as is schematically indicated by 27′l, 27′r. The position of the corresponding light spot from the beams emitted by the indicators 27l, 27r can be adjusted by means of a mechanical drive of the indicators 27l. 27r or, alternatively can be optically adjusted by means of mirrors. According to this technical measure the position of the resulting light spot on the patient's surface can be shifted in compliance with the position of the selected area.

Alternatively, it is possible to use a stationary post-based matrix of pencil light-beams, where the orientation of the resulting fan will be given by a selective energizing of a set of light elements in the matrix in accordance with the spatial position of the scanning plane. An example of such an arrangement is given in FIG. 3 (for simplicity only one unit is being shown). As is shown in FIG. 3, the visualization means 20 comprise a matrix of light emitting elements 26. The visualization means 20 are arranged in an immediate vicinity of the patient support table 2. In order to represent the actual imaging plane, a set of the light-emitting elements 26 are energized as is schematically indicated by squares 28 in FIG. 3. The spatial and angular resolution of such a system depends upon the total number of the light-emitting elements in the matrix 20. By appropriately choosing the total number of the light-emitting elements a simple yet reliable device can be produced.

In order to match the position of the resulting light beam with the position of the imaging plane in the longitudinal direction, the arm or the matrix can be translatably arranged in the longitudinal direction.

FIG. 4 shows schematically an embodiment of the diagnostic apparatus arranged to carry out the method according to the invention. The diagnostic apparatus 1 is a conventional bore-type magnetic resonance apparatus comprising a patient support 2 to position the patient P within the imaging volume 1′ of the diagnostic apparatus 1. In accordance with FIG. 1, the imaging plane of the diagnostic apparatus is defined by a set of gradient fields Gx, Gy, Gz defined by the central control unit 5 and controlled by the control unit 3 of the gradient coils. The control unit 9 of the visualization means 10, 11, 12 is controlled by the central control unit 5 in accordance with the signals sent to the control unit 3 of the gradient coils. The imaging means 6 are arranged to acquire a diagnostic image from the imaging plane defined by the gradient field. The acquired resonance signals from the excited imaging plane are reconstructed using an appropriate reconstruction soft-ware and are graphically represented on the console 7 for the analysis of the operator. At the same time the corresponding imaging plane is being visualized on the surface of the patient P by means of the light fans 10′, 11′, 12′ emitted by the visualization means 10,11,12. A line L representing an intersection of the light fans with the patient's surface unambiguously indicates the spatial position of the actual scanning plane on the patient's surface. In case the patient P is undergoing an interventional procedure it is of a great importance that the interventional apparatus (needle or a medical device) is being introduced into the patient with as little damage to healthy tissue as possible. The accurate selection of the entry point of the interventional apparatus on the patient's skin is ensured due to the fact that the projection of the actual imaging plane comprising a target area is being visualized on the surface of the patient by means of the projection line L. By using the anatomical information contained in the diagnostic image the operator can deduce the correct position of the entry point on the external surface of the patient. By performing an on-line image acquisition during the insertion of the interventional apparatus, the manoeuvering of the latter in an accurate and correct way is ensured.

Alternatively, in case critical tissues are adjacent to the area of a medical intervention, the accurate positioning of the interventional apparatus within the patient can be controlled using a second embodiment of the method of the invention. According to the second embodiment of the method of the invention the operator selects the imaging plane comprising a target area. Then, the operator can interactively define a target point on the target area of the patient. The diagnostic apparatus will then calculate a shortest approach trajectory comprising the target point and an entry point on the surface of the patient, said approach trajectory being preferably a straight line avoiding critical tissues. Such a calculation is performed in the diagnostic apparatus 1 by means of a dedicated computer program stored in the system computer 8. An example of such a computer program is a decision support system (DSS) known in the art of medical application, the program comprising for example tabulated tissue data and corresponding weighing coefficients representing the clinical crucially of the organs. The optimal trajectory is then calculated based on the optimization of the total value function representing a cost function. Optimization methods of the kind are known in the field of combinatorial optimization. After the optimal approach trajectory is calculated, an appropriate signal is sent to the control unit 9 of the visualization means. In case the visualization means, as schematically presented in FIG. 2, are further equipped with an indicator to indicate a selected area on the surface of the patient, said area I being visualized for the operator in accordance with the calculated entry point for the interventional apparatus. The operator is thus provided with an accurate position of the incision point, which is of a great importance especially if the imaging plane is obliquely oriented with respect to the patient. By performing an on-line acquisition of the imaging data during an interventional procedure, the operator can be ensured that the manoeuvering of the interventional apparatus takes place in accordance with the calculated optimal approach trajectory. This technical measure ensures a safe an accurate conduct of the interventional procedures.

While this invention has been described with reference to particular embodiments and examples, other modifications and variations will occur to those skilled in the art in view of the above teaching. Accordingly, it should be understood that within the scope of the appended claims the invention may be practiced otherwise than is specifically described.

1. A diagnostic apparatus comprising an imaging volume for accommodating a patient to be imaged, means for positioning the patient within the imaging volume, imaging means arranged to acquire a diagnostic image in an imaging plane of the patient positioned in the imaging volume, characterized in that the diagnostic apparatus further comprises visualization means arranged to visualize a spatial position of the imaging plane within the imaging volume.

2. An apparatus according to claim 1, characterized in that the visualization means are arranged in the immediate vicinity of the imaging volume and in that the visualization means comprise an adjustable light fan.

3. An apparatus according to claim 2, characterized in that the visualization means further comprise indicators to visualize a selected area within the imaging plane.

4. A method for guiding an interventional apparatus using a diagnostic apparatus according to claim 1, said method comprising the steps of
positioning a patient within the imaging volume of the diagnostic apparatus;
using the imaging means for acquiring a diagnostic image in an imaging plane comprising a target area of the patient;
using the visualization means for visualizing a projection of the imaging plane of said diagnostic image on the patient's skin.

5. A method according to claim 4 characterized in that said method further comprises the step of inserting the interventional apparatus in the patient according to the information contained in the diagnostic image and the imaging plane visualization on the patient's skin.

6. A method for guiding an interventional apparatus using a diagnostic apparatus according to claim 1, said method comprising the steps of:
positioning a patient within the imaging volume of the diagnostic apparatus;
using the imaging means for acquiring a diagnostic image in a plane comprising a target area of the patient;
calculating an approach trajectory for the interventional apparatus, said trajectory comprising an entry point on the patient's skin and a target point within the target area; and
visualizing the entry point together with a projection of the imaging plane (L) of the diagnostic image on the patient's skin.

7. A method according to claim 6, characterized in that said method further comprises the step of inserting the interventional apparatus in the patient according to the calculated approach trajectory, the entry point and the imaging plane visualization on the patient's skin.
